(19) United States
(12) Patent Application Publication
(10) Pub. No.: US 2013/0203711 A1
(43) Pub. Date: Aug. 8, 2013

(54) NOVEL HERBAL OIL FOR CONTROLLING BLOOD SUGAR WITHOUT RISK OF HYPOGLYCEMEA

(71) Applicant: Anil Kumar Gupta, Kanpur (IN)

(72) Inventor: Anil Kumar Gupta, Kanpur (IN)

(21) Appl. No.: 13/821,016

(22) PCT Filed: Oct. 4, 2011
(86) PCT No.: PCT/IN2011/000692

§ 371 Date: Mar. 6, 2013

(30) Foreign Application Priority Data
Oct. 18, 2010 (IN) 1415/DEL/2010

(51) Int. Cl. A61K 31/618; A61K 31/045

(52) U.S. Cl. CPC A61K 31/618; A61K 31/045
USPC 514/164

(57) ABSTRACT

The invention relates to a herbal oil, for controlling blood sugar without risk of hypoglycemia, comprising of the step of (i)
fractional distillation of essential oils comprising of turpentine oil, jasmine oil, rose oil, eucalyptus oil, sandal wood oil and water
green oil to obtain hydroxyl compounds #-terpinol, linalool, Gereneol, Citroneoll, #-Santalol, Methyl salicylate and other alcoholic
terpenoils and (ii) dilution with fat oil so as to have approximate weight percentage of #-terpinol 50%, linalool 6%, Gereneol 4%,
Citroneoll 3.5%, #-Santalol 2.7%, Methyl salicylate 2%, other alcoholic terpenoils and fat oil 31.8%.

### FIELD OF INVENTION

This invention relates to a novel herbal oil for controlling blood sugar without risk of hypoglycemia and is useful in Pharmaceutical industry.

### BACKGROUND OF INVENTION

Diabetes has now become a global disease. Type-II diabetes form 90% of the estimated 40 million diabetic cases in India. Due to diabetes, some time post-meal blood sugar rises high and remain uncontrolled even on taking oral medicines which could lead to a series of complications in brain, kidneys, heart or eyes.

In Type-I diabetes, insulin is injected but this carries the risk of causing hypoglycemia. The instant herbal oil, which could be prescribed for both type of diabetics (Type-I and Type-II), is more cost effective and potent with lesser adverse effects than sulphonamides (Oral drugs) and insulin. Uncontrolled, unchecked highs and lows in blood sugar could be the root cause number of complications in both Type-I and Type-II diabetics.

In Type-I diabetes, insulin is injected but this carries the risk of hypoglycemia (manifested by nervousness, hunger, warmth, sweating headache) and a dangerous condition where the patient can lose consciousness and slip into coma. Insulin causes its hypoglycemic effect by promoting carbohydrate and lipid absorption in peripheral tissues.

Oral drugs, used for the treatment of Type-II diabetes, are substitutes of sulphonamides (sulpha drugs). The hypoglycemic activity of a sulphonamide was first observed by Janbon and colleagues in 1942 and usefulness of carbutamide in the treatment of diabetes mellitus (Type-II) led to the development of sulphonyl ureas—a class of oral hypoglycemic agents.

Sulphonamides (sulpha drugs) are derivatives of sulphanilic acid which contain sulphonamide-SO₂ NH₂ group in the structure. While preparing some synthetic dyes, Glemo was successful in preparing P-aminobenzene sulphonamide in 1908.

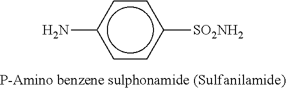

A retrospective look at sulphonamides leave no doubt that besides providing the first efficient treatment of bacterial infections, these unleashed a revolution in chemotherapy by introducing and substantiating the concept of metabolic antagonism which has been very useful in medicinal chemistry to explain the mechanism of action of many drugs and to rationally design new therapeutic agents. With the purpose of obtaining new and hopefully better sulphonamides, to date about 20,000 sulfanilamide derivatives analogs, and related compounds, were synthesized in the most intensive program of molecular modification. Some of these compounds are found to had other pharmacological properties besides, or instead of antibacterial activity.

Further modification of the lead compound with the newly discovered pharmacological activity led to the introduction of many new types of drugs: antibacterial agents (sulphonamide), hypoglycemic agents (sulphonyl ureas).

The general structure of suphonyl urea is

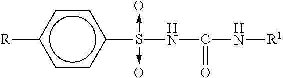

in which R is usually an aliphatic group (mainly acetyl, amino, chloro, methyl, methylthio, trifluoro methyl), and R¹ is either an aliphatic moiety (consisting of three or six carbon atoms) or an alicyclic or hetero cyclic ring. Sulphonyl ureas are rapidly and completely absorbed extensively (70-99%) bound to protein. They are metabolized in the liver to active or inactive compounds that are eliminated in the urine, primarily by tubular secretion.

Hypoglycemia and allergic reactions are the most common adverse effects caused by sulphonamides. Hypoghycemia can cause complications on vital systems of the body, like the brain, kidneys, heart or eyes. Interactions of sulphonyl ureas with other drugs are frequent. β-adrenergic agonists, calcium channel blockers, chlorpromazine, estrogens, glucocorticoids, nicotinic acid, oral contraceptives, Phenobarbital, phenytoin, rifampin, and thiazide increase blood glucose levels. Some drugs decrease blood glucose levels, thus increasing the risk of phyoghycemia in patients treated with sulphonyl ureas, Examples: Alcohol, anabolic steroids, B-blocker's, chloramphemicol, clofibrate, dicumarol, and warfarin.

Owing to their low solubility in water, sulphonamide derivatives can accumulate in the kidneys, producing crystalluria. Other adverse reactions are nausea, dizziness, hyper sensitivity reactions, blood dyscrasias, hepatitis, and several other uncontrolled effects. These drugs are contraindicated for pregnant women and nursing mother and for infants less than 2 months ago.

In the instant invention, a herbal oil is prepared for controlling the highs in blood sugar based on observable, empirical and measurable evidence and also a solution has been found for the problem as to how herbal oil lowers the glucose level by entering into reaction with Glucose indirectly.

The herbal oil has shown promising result in reducing blood sugar to normal level (about 70-100 mg/dl) when measured by glucometer as it enters into the conjugation reaction with the glucoronic acid obtained after conversion of glucose in the liver. While using the herbal oil, the excess of glucose present in blood is converted into more glucoronic acid which then form uridine-di-phosphate-glucoronic acid (UDPAG). Meanwhile free hydroxyl group of pharmacophore present in herbal oil conjugates with UDPAG and form glucuronide in presence of enzyme glucuronyl transferase to reduce glucose level in the blood.

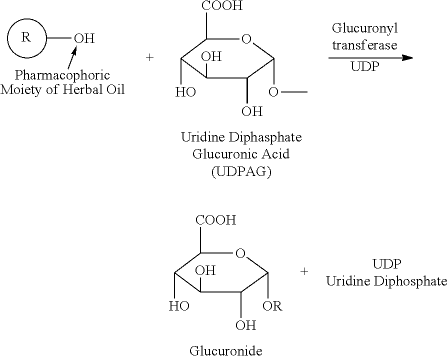

### OBJECTS OF INVENTION

The main object of the invention is to develop a novel herbal oil for controlling blood sugar.

Another object of the invention is to formulate a new herbal oil for controlling blood sugar without risk of hypoglycemia.

Still another object of the invention is to compose a novel herbal oil containing hydroxyl compounds for controlling blood sugar within two hours of application.

Yet another object of the invention is to develop herbal oil by fractional distillation of essential oils having lesser adverse effects than other existing medicines, affecting brain, kidneys, heart or eyes.

Further object of the invention is to prepare a herbal oil composition which is only required when blood sugar level is abnormally high.

An additional object is to develop herbal oil composition that can be used by both type of diabetics (Type-I & Type-II).

The foregoing has outlined some of the pertinent objectives of the invention. These objectives should not be construed to be merely illustrative of some of the more prominent features and applications of the intended invention. Many other beneficial results can be obtained by applying the disclosed invention in a different manner or modifying the invention within the scope of disclosure.

Accordingly, other objectives and a full understanding of the invention and the detailed description of the preferred embodiment in addition to the scope of invention are to be defined by the claims undertaken.

These and other objects and advantages of the invention will be apparent from the ensuing description.

### STATEMENT OF INVENTION

This invention relates to a herbal oil composition in controlling blood sugar without risk of hypoglycemea comprising Hydroxyl compounds ∝ terpineol, linalool, Gereneol, Citronellol, santalol and Mehyl salicylate extracted by distillation of essential oil, terpentine, Jasmine, rose, sandal wood, eucalyptus and winter green oil having approximate Wt. % composition of the herbal oil, α-terpineol 50%, Linalool 6% Gereneol 4%, Citronellol 3.5%, α-Santalol 2.7%, Methyl Salicylate 2%, Other alcoholic terpenoids and fat oil 31.8%, wherein two ml of the oil which is massaged contains approximately α-terpineol 1 gm., linalool 0.12 gm., gereneol 0.08 gm., citronellol 0.07 gm., α-santalol 0.054 gm., methyl salicylate 0.04 gm. and other alcoholic terpenoids and fatoil 0.64 grams.

### DETAILED DESCRIPTION OF INVENTION

At the outset of the description, which follows, it is to be understood that the ensuing description only illustrates a particular form of this invention. However, such a particular form is only an exemplary embodiment, and without intending to imply any limitation on the scope of this invention. Accordingly, the description is to be understood as an exemplary embodiment and teachings of the invention are not intended to be taken restrictively.

For the purpose of promoting an understanding of the principles of the invention, reference is now to be made to the embodiment illustrated in the description and specific language is used to describe the same. It is nevertheless to be understood that no limitations of the scope of invention is hereby intended, such alterations and further modifications in the illustrated bag and such further applications of the principles of the invention as illustrated therein being contemplated as would normally occur to one skilled in the art to which the invention relates.

The present invention illustrates a novel herbal oil which has been developed using turpentine oil, jasmine oil, rose oil, eucalyptus oil, sandal wood oil and water green oil. These essential oils are fractionally distilled to have hydroxyl compounds α-terpinol, linalool, Gereneol, Citroneoll, α-Santalol, Methyl salicylate and other alcoholic terpenoils & fat oil and then diluted with fat oil so as to obtain approximate weight percentage of α-terpinol 50%, linalool 6%, Gereneol 4%, Citroneoll 3.5%, α-Santalol 2.7%, Methyl salicylate 2% and other alcoholic terpenoils and fat oil 31.8%.

The instant herbal oil is never consumed orally. 2 ml. of the herbal oil can be massaged at a time and its amount to be used could be monitored according to the amount of fasting plasma glucose and post prandial plasma glucose measured in the blood.

When the oil is massaged near liver, the drug is absorbed through the skin into the blood supply and passes via the blood stream to the liver. In the lever, the drug undergo the metabolic transformation to become hydrophilic and less lipophilic. After the metabolism in the liver, the metabolite passes from liver into bile and hence via the gall bladder goes to the gastro intestinal tract to be further metabolized by gut microflora (Billary Excretion).

This process of cycling via the bile is termed as enterohepatic circulation and explains the commonly observed phenomenon that a drug given other than orally can get eliminated in the faeces².

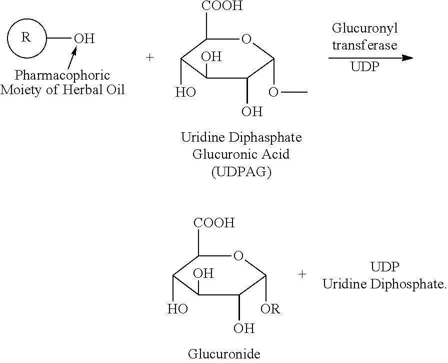

The instant herbal oil for controlling glucose level in diabetic patients is found to be safest medicine in comparison to insulin and sulphonamides as glucuronide is hydrolysed in gut in case sugar level is droped below normal level 70-100 mg/dl to give back glucoronic acid resulting into a further conversion of glucose to glucoronic acid is immediately stopped. Thus the risk of hypoglycemia is prevented.

It is evident from the following Table A that as long as herbal oil is massaged into the body of patient, the value of fasting plasma glucose and post prandial plasma glucose decreases within two hours:

TABLE A
Amount of fasting and post prandial plasma glucose before and
after massage of herbal oil

| | Before Massage | | After Massage | |
|---|---|---|---|---|
| | Fasting | | Fasting | |
| | plasma | Post Prandial | plasma | Post prandial |
| Month/ | glucose in | plasma glucose | glucose in | plasma |
| Year | mg./dl | in mg./dl | mg./dl | glucose |
| 6/05 | 170 | 230 | 75 | 102 |
| 9/05 | 157 | 213 | 72 | 100 |
| 10/07 | 206 | 336 | 86 | 216 |
| 6/08 | 146 | 275 | 76 | 155 |
| 4/07 | 185 | 297 | 105 | 177 |
| 10/08 | 193 | 279 | 113 | 159 |
| 6/09 | 171 | 270 | 91 | 150 |
| 1/10 | 190 | 280 | 110 | 160 |

The value of fasting plasma glucose ultimately attains lowest value near around 70-100 mg/dl when measured by glucometer but not less than around 70 mg/dl, this is be because glucoronide is not stable below this level of glucose, and is hydrolysed back to give glucoronic acid and pharmacophore to be eliminated in the faeces. Glucuronide hydrolysis also stops the process of conversion of glucose into glucoronic acid in the liver and thus prevents any further possibility of hypoglycemic condition.

The study revealed, a fall of nearly 120 mg/dl of post prandial plasma glucose in approximately two hours after the massage of 2 ml. of oil and, if the quantity of the oil is reduced to half i.e. one ml. then nearly 60 mg/dl., of the post prandial plasma glucose is reduced. The value of post prandial plasma glucose ultimately attains lowest value near around 100-140 mg/dl but not less than 100 mg/dl due to unstability of glucuronide below this amount.

### Composition Constituents

The herbal oil is a mixture of hydroxyl compounds namely α-terpineol, linalool, geraniol, cetronellol, santalol and methyl salicylate, which are present in essential oils used in this invention namely terpentine, Jasmine, rose, sandal wood, Eucalyptus oil and wintergreen oil.

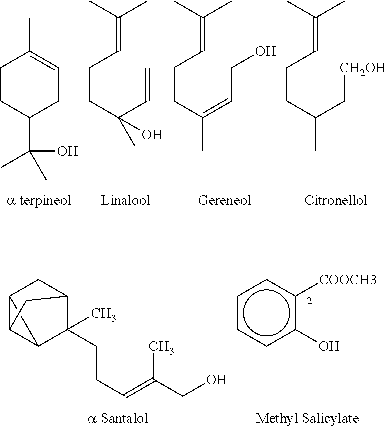

OH group of different hydroxy compounds conjugates with the UDPAG as evident from the example of methyl saliscylate in presence of enzyme glucuronyl transferase and reduce the glucose level in blood more powerfully.

The instant herbal oil comprises of

| Constituents | Wt. % (Approx.) |
|---|---|
| 1. α-terpineol | 50.0 |
| 2. Linalool | 6.0 |
| 3. Gereneol | 4.0 |
| 4. Citronellol | 3.5 |
| 5. α-Santalol | 2.7 |
| 6. Methyl Salicylate | 2.0 |
| 7. Other alcoholic | 31.8 |
| terpenoids & fat oil | |

2 ml of the oil which is massaged contains approx.:

| | |
|---|---|
| α-terpineol | 1.00 gm., |
| linalool | 0.12 gm., |
| gereneol | 0.08 gm., |
| citronellol | 0.07 gm., |
| α-santalol | 0.054 gm., |
| methyl salicylate | 0.04 gm. and |
| other alcoholic | 0.64 grams. |
| terpenoids & fat oil | |

### Process of Preparation:

A mixture of:
60 ml. terpentine oil,
20 ml. jasmine oil,
10 ml. rose oil,
5 ml. eucalyptus oil,
3 ml. sandal wood oil and
2 ml. winter green oil,

is to be boiled in round bottom flask at 300° C. using water bath and water condenser. Mixture of hydroxy compounds were extracted one by one at their respective boiling points in the receiver and then diluted up to 100 ml. by fat oil. The determination of composition of the herbal oil was done by newly invented acetylation method.

To summarise, a novel herbal oil has been developed which is best suited for controlling the blood sugar without any risk of hypoglycemea.

All documents cited in the description are incorporated herein by reference. The present invention is not intended to be limited in scope by the specific embodiments and examples which are intended as illustration of a number of aspects of the scope of this invention. Those skilled in art will know or to be able to ascertain using no more than routine experimentations many equivalents to the specific embodiments of the invention described herein.

It is to be further noted that present invention is susceptible to modifications, adaptations and changes by those skilled in the art. Such variant embodiments employing the concepts and features of this invention are intended to be within the scope of the present invention which is further set forth under the following claims:

### Advantages:

1. Though there are other medicines available in the market in the form of substituted sulphonamides and insulin but these medicines can lead to series of complications in brain, kidneys, heart or eyes.
2. The result of herbal oil become visible within two hours of application.
3. Other existing medicines have binding to be taken daily but herbal oil is only required when blood sugar level is abnormally high.
4. The herbal oil is more potent with lesser adverse effects than other existing medicines.
5. The herbal oil can be used by both type of diabetics (Type I & Type II).

I claim:

1. A herbal oil, for controlling blood sugar without risk of hypoglycemia, comprising of the step of:
(i) Fractional distillation of essential oils comprising of turpentine oil, jasmine oil, rose oil, eucalyptus oil, sandal wood oil and water green oil to obtain hydroxyl compounds α-terpinol, linalool, Gereneol, Citroneoll, α-Santalol, Methyl salicylate and other alcoholic terpenoils and
(ii) dilution with fat oil so as to have approximate weight percentage of α-terpinol 50%, linalool 6%, Gereneol 4%, Citroneoll 3.5%, α-Santalol 2.7%, Methyl salicylate 2%, other alcoholic terpenoils and fat oil 31.8%.

2. A herbal oil, for controlling blood sugar without risk of hypoglycemia, as claimed in claim 1, wherein two ml of such oil, which is massaged, contains approximately α-terpineol 1 gm., linalool 0.12 gm., gereneol 0.08 gm., citronellol 0.07 gm., α-santalol 0.054 gm., methyl salicylate 0.04 gm. and other alcoholic terpenoids and fat oil 0.64 grams.

3. A herbal oil, as claimed in claim 1, used for controlling blood sugar without risk of hypoglycemia, as described and illustrated herein.
